(19) United States
(12) Patent Application Publication
(10) Pub. No.: US 2023/0003655 A1
(43) Pub. Date: Jan. 5, 2023

(54) ARTIFICIAL INTELLIGENCE METHODS FOR CORRELATING LASER-INDUCED BREAKDOWN SPECTROSCOPY (LIBS) MEASUREMENTS WITH DEGREE OF SENSITIZATION (DOS) VALUES TO DETERMINE THE SENSITIZATION OF AN ALLOY

(71) Applicant: NUtech Ventures, Lincoln, NE (US)

(72) Inventors: Yongfeng Lu, Lincoln, NE (US); Lei Liu, Lincoln, NE (US); Xi Huang, Lincoln, NE (US)

(21) Appl. No.: 17/890,955

(22) Filed: Aug. 18, 2022

Related U.S. Application Data
(63) Continuation of application No. PCT/US2021/019860, filed on Feb. 21, 2021.
(60) Provisional application No. 62/981,952, filed on Feb. 26, 2020.

(51) Int. Cl. G01N 21/71; G01J 3/443; G01N 33/202; G01N 1/32

(52) U.S. Cl. CPC G01N 21/718; G01J 3/443; G01N 33/202; G01N 1/32

(57) ABSTRACT

Methods and systems for determining sensitization of an alloy includes correlating laser-induced breakdown spectroscopy (LIBS) measurements with degree of sensitization (DoS) values to determine the sensitization of an alloy. Sensitization is characterized by new phase precipitates preferably along the grain boundaries (GBs). In an embodiment, the method includes the features of (1) selective chemical etching of the new phase precipitate of an alloy to induce quantitative chemical composition change, correlated with the DoS values, on the alloy surface, (2) LIBS measurements to semi-quantitatively probe the chemical composition change on the etched surface due to selective chemical etching, (3) establishing calibration models by correlating the LIBS spectra with the DoS using artificial intelligence (AI) algorithms/approaches to determine a sensitization of an alloy.

### CROSS REFERENCE TO RELATED APPLICATION

100911 This Patent Application is a continuation of International Application No. PCT/US2021/019860, filed Feb. 21, 2021, which claims priority to U.S. Provisional Patent Application No. 62/981,952, filed Feb. 26, 2020, both of which are entitled “ARTIFICIAL INTELLIGENCE METHODS FOR CORRELATING LASER-INDUCED BREAKDOWN SPECTROSCOPY (LIBS) MEASUREMENTS WITH DEGREE OF SENSITIZATION (DOS) VALUES TO DETERMINE THE SENSITIZATION OF AN ALLOY”, and both of which are incorporated herein by reference in their entireties.

### FIELD

The present embodiments relate to methods for determining sensitization of an alloy by correlating laser-induced breakdown spectroscopy (LIBS) measurements with the degree of sensitization (DoS) using various approaches, including Artificial Intelligence (AI) approaches. The present embodiments have significant engineering applications for nondestructive onsite sensitization assessment of metal alloys (such as aluminum alloys) in various industries such as transportation (maintaining of ships, airplanes, vehicles, oil/gas pipelines), nuclear (maintaining of nuclear power station), construction (maintaining of steel structure, bridge, and facility), and metallurgical engineering (Al alloy manufacture and treatment) industries.

### BACKGROUND

An alloy is generally comprised of a matrix metal element mixed with one or more other elements. Generally, an alloy is a polycrystalline material with the interface among solid crystallites of the alloy as the grain boundaries (GBs). Sensitization is characterized by the formation of a new phase precipitate preferably along the GBs due to migration of specific atoms when exposed to elevated temperature for a period of time, which causes the GBs to have different physical and chemical properties from the homogenous alloy and to be susceptible to intergranular corrosion (IGC) and stress corrosion cracking (SCC). The general alloys susceptible to sensitization includes the aluminum alloys and stainless steel.

Aluminum (Al) alloys are extensively used in marine transportation and military applications due to their high strength-to-weight ratio, formability, cold workability as well as weldability. For example, the 5xxx series magnesium-aluminum (Mg—Al) alloys provide the combined properties of high strength-to-weight ratio and excellent corrosion resistance, and therefore, are often used as the primary structural material for building high-speed ships and vessels in marine transportation or military applications. The magnesium (Mg) alloying element is used to strengthen the original aluminum material by solid solution strengthening. However, these materials are susceptible to sensitization over time after exposure to elevated temperature.

Sensitization of the 5xxx alloys is characterized by the formation of precipitates primarily along the material grain boundaries (GBs), such as the magnesium rich β-phase (Mg₂Al₃, ˜Mg 37.5 wt %) precipitates in 5xxx alloys. When exposed to harsh environments in service, such as sea water, a galvanic coupling is formed between the aluminum matrix and the β-phase precipitates, leading to preferential dissolution of these precipitates and resulting in IGC cracking. Sensitization decreases the mechanical and chemical properties of the material and increases the susceptibility to IGC and stress corrosion cracking (SCC) at moderate or even low temperatures (65° C.) [1]. The 5xxx aluminum alloys were used as the critical construction materials in the design and construction of state-of-the-art Navy ships [2]. Sensitization caused material degradation is currently one of the most important problems that is costly and needs to be taken care of. Therefore, technologies for rapid and nondestructive onsite characterization of aluminum alloy sensitization are highly desired to improve the work efficiency and reduce the total ownership cost.

A few methodologies have been reported for characterization of the degree of sensitization (DoS) in metal alloys, including the American Society for Testing and Materials (ASTM) standards, ultrasonic methods, metallographic imaging, X-ray diffraction (XRD), eddy current testing (ECT), and electrochemical methods [2-8]. These techniques have their own advantages and drawbacks with most of them being laboratory-based technologies or to be improved for onsite applications with efficiency and economy [3-7]. Specifically for the 5xxx alloys, the standard method for quantification of the DoS is the ASTM G67 nitric acid mass loss test (NAMLT) technique which uses the mass loss of the material after immersed in concentrated nitric acid for 24 hours to quantify the DoS values [8]. The DoS value is the amount of mass loss of the alloy in unit of mg/cm² calculated from the ASTM G67 test. This method is quantitative and accurate but needs samples to be cut from material, which will damage the related structures. Also, this technology is also a laboratory-based technology as well as time consuming. Some other reported techniques for characterizing the DoS levels have their own drawbacks for onsite applications [3-5]. One currently available technique used for onsite testing of sensitization on ships is the electrochemical method (also referred as the DoS probe), which utilizes electrochemical measurements of the relationships between the current and time to quantify the DoS levels. However, this technology needs surface polishing (e.g., 4″ diameter to 1200 grit), test area heating to up to 50° C., a typical measurement time of 75 min, and expensive maintenance [2]. Therefore, there is still an expectation and need for developing field portable technologies for onsite characterization of an alloy sensitization with the characteristics of being fast, cheap, and flexible to be calibrated for multiple alloys.

### SUMMARY

The present embodiments provide methods for determining sensitization of an alloy by correlating the laser-induced breakdown spectroscopy (LIBS) measurements with the degree of sensitization (DoS) to determine the sensitization of an alloy. The various embodiments advantageously enable nondestructive onsite sensitization assessment of metal alloys in various industries. The chemical etching and LIBS measurements involved may only slightly affect the alloy surface within sub-millimeter in depth and sub-square centimeter in area. Hence, the methods induce no influences on the structural integrity and can advantageously be characterized as nondestructive. The various embodiments may include the following features: (1) selective chemical etching of the new phase precipitate of an alloy to induce quantitative chemical composition change, correlated with the DoS values, on the alloy surfaces. Generally, the new phase precipitate is physically and chemically different from the homogeneous alloy. In addition, the amount of new phase precipitates positively correlates with the DoS. Therefore, selective chemical etching of the new phase precipitate induces different quantitative chemical composition changes on the etched alloy surfaces, which correlates with the DoS of the metal alloy, e.g., selective etching of the β-phase on the 5xxx Al—Mg alloy surfaces results in the residual Mg concentration negatively correlating with the DoS; (2) LIBS measurements to semi-quantitatively probe the chemical composition change on the etched surfaces, e.g., a single pulse LIBS system with gating measurements may be used to semi-quantitatively provide the chemical composition of the locally laser-ablated material; (3) establishment of calibration models by correlating the LIBS spectra with the DoS using artificial intelligence (AI) approaches to determine sensitization of an alloy, e.g., a statistical method of principal component and discriminant function analysis (PC-DFA) may be used to establish a calibration model by correlating the LIBS spectra with the DoS values to determine sensitization of the alloy, e.g., 5456 Al—Mg alloy, from LIBS spectra unknown to the model.

According to an embodiment, a method is provided for determining sensitization of an alloy. The method includes selective chemical etching a surface of the alloy, wherein the selective chemical etching is performed using one or more alloy etchants, measuring laser-induced breakdown spectroscopy (LIBS) spectra of the etched surface of the alloy using a LIBS system to semi-quantitatively probe a chemical composition change of a new phase precipitate of the alloy on the etched surface of the alloy due to the selective chemical etching, and correlating the LIBS spectra with the degree of sensitization (DoS) of the alloy to thereby determine a sensitization of the alloy. In an embodiment, the correlation includes using an artificial intelligence (AI) algorithm to thereby determine the sensitization of the alloy.

According to another embodiment, a method is provided for determining sensitization of an alloy by correlating laser-induced breakdown spectroscopy (LIBS) measurements with the degree of sensitization (DoS) using artificial intelligence (AI). The method includes selective chemical etching of the new phase precipitate of an alloy to induce quantitative chemical composition change on a surface of the alloy, wherein the selective chemical etching is conducted using one or more alloy etchants. The method also includes measuring LIBS spectra using a LIBS system to semi-quantitatively probe the chemical composition change on the etched surface of the alloy, wherein the LIBS spectra are measured using a single laser pulse or multiple laser pulses, and determining a sensitization of the alloy using an artificial intelligence (AI) algorithm, wherein the AI algorithm correlates the LIBS spectra with the DoS.

In certain aspects, the alloy is an aluminum alloy or a steel, such as a stainless steel. In certain aspects, a new phase is formed by migration of atoms of specific elements in a crystalline material and is different from the homogeneous alloy. In certain aspects, the one or more alloy etchants include one or more of nitric acid, Keller's reagent, and ammonium persulfate. In certain aspects, a method may include surface polishing the surface of the alloy before the selective chemical etching. In certain aspects, the surface polishing includes one of sanding, ultrasonic polishing, lapping, sandblasting, rumbling and tumbling.

In certain aspects, the LIBS system includes one or more pulsed lasers, one or more compact or bulk spectrometers, and optical components for light delivery and collection, including for example reflectors, beam splitters, lens, optical filters, and/or optical fibers. In certain aspects, the LIBS system includes a single-pulsed laser beam as the plasma excitation source. In certain aspects, the LIBS system includes a double-pulse or multiple-pulse laser beam as the plasma excitation source.

According to an embodiment, a laser-induced breakdown spectroscopy (LIBS) system for determining sensitization of an alloy is provided. The system includes a pulsed laser source configured to emit laser pulses directed at a sample to induce a plasma on a surface of the sample, the sample including a selectively chemical etched surface of an alloy, a spectrometer configured to measure LIBS spectra of the etched surface of the alloy and/or the plasma to semi-quantitatively probe a chemical composition change of a new phase precipitate of the alloy on the etched surface of the alloy, and one or more processors, configured to receive the LIBS spectra and to correlate the LIBS spectra with a degree of sensitization (DoS) of the alloy to thereby determine a sensitization of the alloy. DoS values may be stored to, and retrieved from, a memory or storage medium coupled with the one or more processors. The LIBS spectra may be stored to, and retrieved from, a memory or storage medium coupled with the one or more processors. All values, e.g., DoS values, LIBS spectra and sensitization may be output or may be displayed on a display device coupled with the one or more processors. In certain embodiments, a non-transitory computer-readable medium is provided to store code, which when executed by one or more processors, cause the one or more processors to execute any method as described herein, e.g., to execute correlation algorithms and/or to control one or more system components of the LIBS system.

In certain aspects, the one or more processors are configured to correlate using an artificial intelligence (AI) algorithm to determine the sensitization of the alloy. In certain aspects, the one or more processors control synchronization, gating and timing parameters of the spectrometer and/or laser source, e.g., triggering and synchronization of the spectrometer with a pulsed laser for gating measurements.

In certain aspects, the pulsed laser source includes a single pulse laser source or a multiple pulse laser source. In certain aspects, the LIBS system further includes one or optical components, e.g., reflectors, lenses, prisms, etc., for light delivery and collection.

In certain aspects, gating measurements are used. For example, the plasma emission is detected during a defined gating interval, with synchronization of each laser pulse. The gating interval parameters of gate delay may be between about 1 ns to about 1 ms and the gate width may be larger than about 1 ns. In certain aspects, non-gating measurements are used. For example, the plasma emission is detected during an integrated time period, without synchronization of each laser pulse. The integrated time period may be larger than about 1 ns.

In certain aspects, a method may further include implementing a LIBS signal enhancement approach, wherein the LIBS signal enhancement approach includes one of spatial confinement, magnetic confinement, flame enhancement, and argon gas enhancement.

In certain aspects, the DoS conforms to the standards set by ASTM international standard G67-18. In certain aspects, the DoS conforms to the standards set by ASTM international standard G108-94 (2015).

In certain aspects, determining sensitization includes providing the quantitative information of the DoS within a range or with a specific value of an alloy.

In certain aspects, the artificial intelligence algorithm includes one of principal component-discrimination function analysis (PC-DFA), discriminant analysis, partial least squares regression analysis, partial least squares discriminant analysis, a k-nearest neighbors algorithm, an artificial neural network, soft independent modeling of class analogy, support vector machines, and classification and regression trees. In certain aspects, the artificial intelligence algorithm includes one of statistical learning, computer intelligence, and soft computing.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE DRAWINGS

Embodiments will be described in conjunction with the appended drawings to illustrate and not to limit the embodiments, and in which:

FIG. 1 is the optical microscope images of the etched surfaces of sensitized 5456 alloy correlating with the DoS of (a) 7.1, (b) 20.2, and (c) 47.3 mg/cm², respectively.

FIG. 2 is a schematic illustrating aspects of an exemplary embodiment of an apparatus for LIBS.

FIG. 3 is the LIBS spectra measured from the etched surfaces of the 5456 alloy correlating with different DoS values.

FIG. 4 is the scatterplots of the PC-DFA of the training and test data correlating with different DoS values of the 5456 alloy.

### DETAILED DESCRIPTION

The aspects and features of the present embodiments summarized above can be embodied in various forms. The following description shows, by way of illustration, combinations and configurations in which the aspects and features can be put into practice. It is understood that the described aspects, features, and/or embodiments are merely examples for purposes of explanation, and that one skilled in the art may utilize other aspects, features, and/or embodiments or make structural and functional modifications without departing the scope of the present disclosure.

The present embodiments provide systems and methods to determine the sensitization of an alloy by correlating the LIBS measurements with the DoS. Certain embodiments use artificial intelligence (AI) approaches. LIBS is an optical emission spectroscopy technique used for chemical composition analysis. Characteristics of LIBS include real-time in-situ analysis, no sample preparation, nearly non-destructive, multi-element analysis, remote detection, and a typical limit of detection (LoD) down to a few ppm. In LIBS measurements, a plasma is generally induced via breakdown of the ablated material by pulsed lasers. The excited atoms/ions transition to lower energy levels and emission chemical element characteristic photons during the plasma cool-down process. The optical emission from the plasma is collected and coupled into spectrometers for spectroscopic measurements. Hence, LIBS measurements semi-quantitatively provide the information of chemical composition of the material with the typical LoD down to a few ppm and are used for chemical analysis of an alloy in the present embodiments.

Generally, the amount of the new phase precipitates positively correlates with the DoS values. Selective chemical etching of the new phase precipitate induces quantitative chemical composition change on the etched alloy surfaces, which is correlated with the DoS values and can be probed by LIBS. Quantitative correlations between the LIBS measurements and the DoS values can be established using AI approaches/algorithms and can be used to determine the sensitization of an alloy. Hence, correlating the LIBS measurements from the selectively etched alloy surfaces with the DoS values provides a method to determine sensitization of an alloy. For purposes of illustration, the 5456 Al—Mg alloy is used as an exemplary alloy to illustrate an embodiment. More specifically, there are generally more β-phase (e.g., Mg₂Al₃, with Mg˜37.5 wt %) precipitates along the GBs for the 5xxx exemplary alloys with higher DoS values. Selective chemical etching of the β-phase causes a negative correlation between the residual Mg concentration on the etched surfaces and the DoS values. Generally, the Al—Mg alloy is considered as unsensitized with a DoS value of 15 mg/cm² or less, indeterminate with a DoS value within the range of 15-25 mg/cm², and sensitized with a DoS value greater than 25 mg/cm² [1, 9]. Determination of the material DoS level as unsensitized, indeterminate, or sensitized is important to support modernization planning, repair and maintenance activities.

The present embodiments provide systems and methods for determining sensitization of an alloy by correlating the LIBS measurements with the DoS to determine the sensitization of an alloy. The embodiments advantageously enable nondestructive onsite sensitization assessment of metal alloys in the transportation industry, nuclear industry, construction industry, and metallurgical engineering industry, among others. More specifically, in embodiments, selective chemical etching induced quantitative chemical composition change, correlating with the DoS, on the etched alloy surfaces may be probed by LIBS. Artificial intelligence (AI) analysis of the chemical composition change due to measurements of selectively chemical etched surfaces are correlated with the DoS to establish calibration models to determine the sensitization of an alloy. The selective chemical etching and LIBS measurements involved may slightly affect the alloy surface within sub-millimeter in depth and sub-square centimeter in area. Hence, the methods induce no influences on the structural integrity and can be characterized as nondestructive.

According to an embodiment, a method includes the following features (using the 5456 Al—Mg alloy as an example for purposes of illustration):

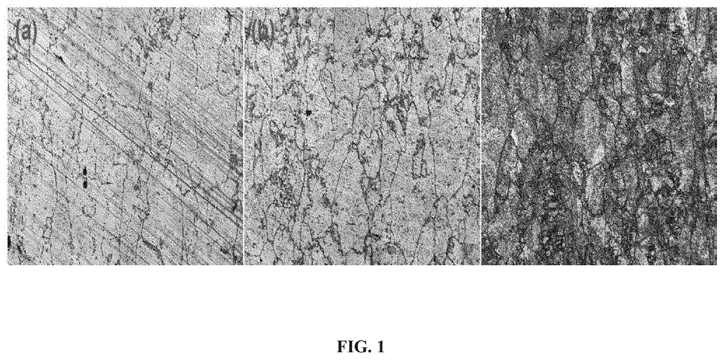

(1) Selective chemical etching of the new phase precipitate of an alloy to induce quantitative chemical composition concentration change, correlated with the DoS values, on the alloy surface. Take the 5xxx aluminum alloy as an example, the β-phase (Mg₂Al₃, Mg˜37.5 wt %) in the 5456 Al—Mg alloy is chemically different from the homogeneous alloy (Mg˜5 wt %). In addition, the amount of β-phase (Mg₂Al₃) precipitates positively correlates with the DoS. Therefore, selective chemical etching of the β-phase precipitates induces different amounts of Mg concentration change on the etched alloy surfaces, with the residual Mg concentration on the etched surfaces negatively correlating with the DoS. FIGS. 1(a) to 1(c) show laser microscope images of an etched surfaces of a 5456 alloy correlating with the DoS values of 7.1 (unsensitized), 20.2 (indeterminate), and 47.3 (sensitized) mg/cm², respectively. The DoS values of the used alloys were identified by the ASTM G67-13 nitric acid mass loss test (NAMLT). Before the chemical etching, the sample surfaces were polished, e.g., using sandpapers to 800-grit. The polished surfaces were then immersed into nitric acid (70%) for 2 hours at room temperature for selective β-phase etching.

(2) LIBS measurements to semi-quantitatively probe the chemical composition change on the etched surface due to selective chemical etching. For example, a single pulse LIBS system with gating may be used for measurements to probe the chemical composition of the laser locally ablated material. It should be appreciated that a single pulse LIBS system or a multiple pulse LIBS system may be used, with or without gating.

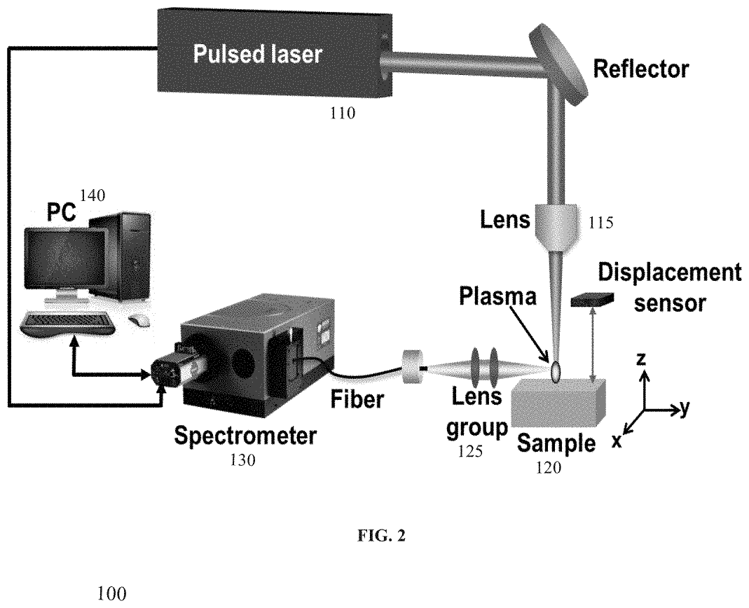

FIG. 2 shows a schematic illustrating aspects of an exemplary embodiment of an LIBS system 100. A pulsed laser beam is generated by pulsed laser beam source 110 and focused onto the target surface 120 (e.g., “sample”) by an optical lens 115 to locally ablate the material and induce plasmas on the target surface. The excited atoms/ions in the plasma transit to lower energy levels during plasma cooling and emit chemical element characteristic photons, the light of which is collected by an optical collection element 125, which may include one or multiple lenses, such as a pair of lenses, and coupled into a spectrometer 130 via an optical fiber for spectroscopic measurements. The spectrometer 130 communicates with a computer system 140 including one or more processors and associated memory and storage for data storage and data processing. A LIBS spectrum provides semi-quantitative information of the chemical composition of the material locally ablated by the laser. The sample may be held by a motor controlled 3-axis stage (not shown) for area scanning, e.g., to provide a fresh surface for each laser pulse. A laser displacement sensor may be fixed above the sample surface to assure consistent focal distance for all samples by tracking the position of sample surfaces. In an embodiment, the spectrometer 130 is externally triggered and synchronized with the pulsed laser for gating measurements, with the synchronization moment as the gate delay time zero. A gate delay and gate width of 0.3 μs and 5 μs, respectively, were used for LIBS measurements in an example.

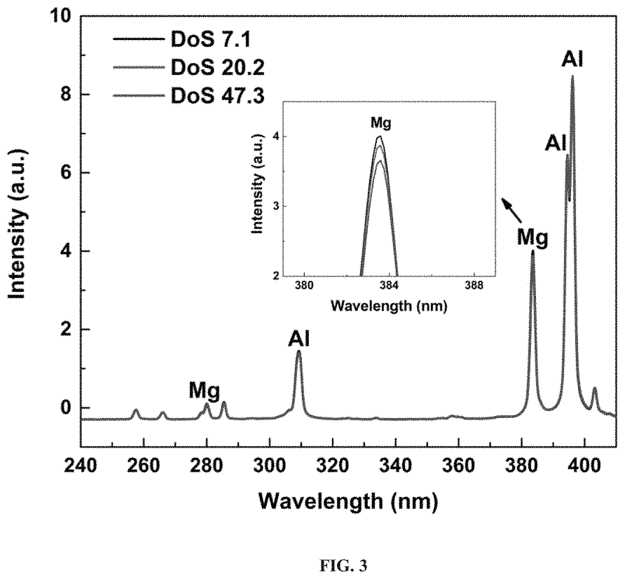

The semi-quantitative chemical composition information on the etched surfaces correlating with different DoS values were probed by LIBS. FIG. 3 shows the LIBS spectra, each normalized and averaged from 2000 laser pulse measurements, measured from the etched surfaces correlating with the DoS values of 7.1 (black line), 20.2 (red line), and 47.3 (blue line) mg/cm², respectively. As shown in FIG. 3, peak intensities of the emission line of Mg I 383.5 nm (a combination from Mg I 382.9, 383.2, and 383.8 nm) negatively correlates with the DoS values, indicating less residual Mg concentration on the etched surfaces with higher DoS values. This is consistent with the material property change as described in selective chemical etching that the residual Mg concentration negatively correlates with the DoS of the 5xxx Al—Mg alloys after selective β-phase etching. The quantitative chemical composition change due to selective chemical etching was successfully probed by LIBS since LIBS has a typical LoD down to a few ppm.

(3) Establishment of calibration models by correlating the LIBS spectra with the DoS, e.g., using artificial intelligence (AI) approaches/algorithms, to determine sensitization of an alloy. For example, a statistical method of principal component and discriminant function analysis (PC-DFA) was used to establish a calibration model by correlating the LIBS spectra with the DoS values to determine sensitization of the 5456 Al—Mg alloy from LIBS spectra unknown to the model. In a specific example, to establish a calibration model and validate the model performance, 400 LIBS spectra correlating with each DoS value were used for data training and prediction, with 350 of them used for data training and the remaining 50 used for prediction test. The statistical multivariate analysis method of PC-DFA was used for data training and prediction. Before the PC-DFA, all LIBS spectra within the wavelength range of 240-400 nm were pre-processed by a standard normal variate technique without background removal to minimize multiplicative error. In PC-DFA, principal component analysis (PCA) was firstly performed on the LIBS spectra to reduce the data dimensions into principal components (PCs) which were then used as inputs for discriminant function analysis (DFA). The PCs sequentially carry the most important information of the dataset, whereas DFA maximize the separation among groups while minimizing variation within group.

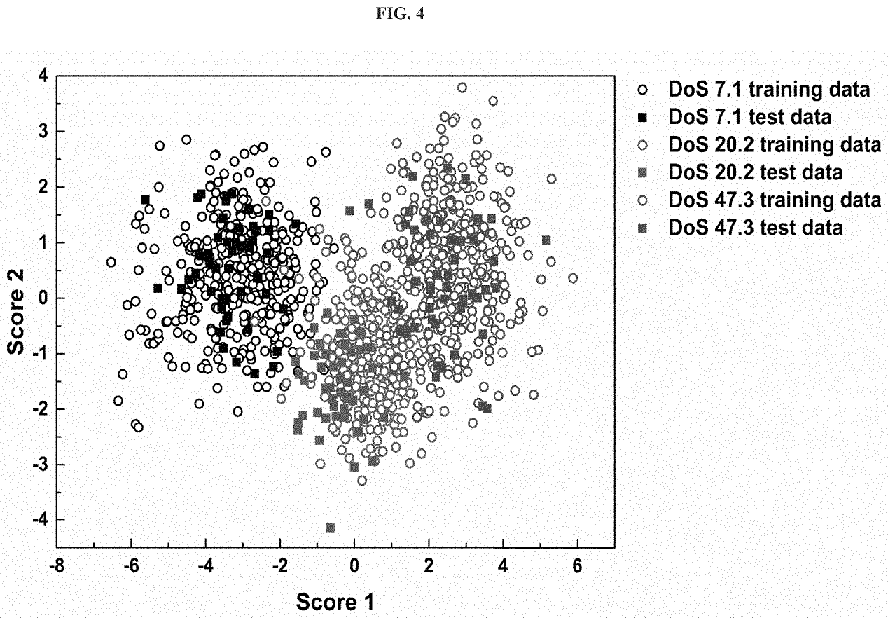

In this exemplary study, the initial 10 PCs were retained for DFA, altogether carrying 87.95% of the most important information of the dataset. FIG. 4 shows the scatterplots of the first two PCs of the PC-DFA of the training (open circular symbols, 350 LIBS spectra correlating with each DoS value) and test (solid square symbols, 50 LIBS spectra correlating with each DoS value) data correlating with different DoS values of the 5456 alloy. The training data correlating with different DoS values were correctly separated in PC-DFA with an accuracy of 94.2%. The performance of the established calibration model was then evaluated by an external validation method for DoS determination. The test data were unknown to the established model. As shown in FIG. 4, the established calibration model accurately assigned the test data to their correlating DoS groups with the correct assignment probabilities of 100%, 100% and 82%, respectively, for the DoS groups of 7.1, 20.2, and 47.3 mg/cm². (See, Table 1, below) Therefore, the test data were accurately determined to correlate DoS groups with high confidence, demonstrating the feasibility of the method for determining sensitization of an metal alloy.

TABLE 1
Results of external validation of multivariate calibrated model for DoS determination.

| Test data correlated | | | | | |
|---|---|---|---|---|---|
| with DoS levels | No. of test | Prediction results | | | Identification of |
| (mg/cm²) | data | Unsensitized | Indeterminate | Sensitized | DoS levels |
| Unsensitized (7.1) | 50 | | 0 | 0 | Unsensitized |
| Indeterminate (20.2) | 50 | 0 | 100% | 0 | Indeterminate |
| Sensitized (47.3) | 50 | 0 | 18% | 82% | Sensitized |

All references, including publications, patent applications, and patents, cited or discussed herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the disclosed subject matter (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or example language (e.g., “such as”) provided herein, is intended merely to better illuminate the disclosed subject matter and does not pose a limitation on the scope of the invention unless otherwise claimed. No language in the specification should be construed as indicating any non-claimed element as essential to the practice of the invention.

Certain embodiments are described herein. Variations of those embodiments may become apparent to those of ordinary skill in the art upon reading the foregoing description. The inventors expect skilled artisans to employ such variations as appropriate, and the inventors intend for the embodiments to be practiced otherwise than as specifically described herein. For example, the formation of the beta-phase may occur anywhere, and not just at the grain boundaries, in a sample. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

### REFERENCES

1. Golumbfskie, W. J., et al., Survey of Detection, Mitigation, and Repair Technologies to Address Problems Caused by Sensitization of Al-Mg Alloys on Navy Ships. Corrosion, 2016. 72(2): p. 314-328.
2. Dunn, R., Quantitative Nondestructive 5XXX Aluminum Material Assessments to Reduce Total Ownership Cost. Naval Engineers Journal, 2016. 128(1): p. 23-34.
3. Holtz, R. L. and D. Horton, Surface metallographic method for characterizing the degree of sensitization of aluminum-magnesium alloys. 2018, Google Patents.
4. Cobb, A., et al. Detecting sensitization in aluminum alloys using acoustic resonance and EMAT ultrasound. in AIP Conference Proceedings. 2017. AIP Publishing.
5. Li, F., et al., Measurements of degree of sensitization (DoS) in aluminum alloys using EMAT ultrasound. Ultrasonics, 2011. 51(5): p. 561-570.
6. Kim, H. J., et al., Investigation of the sensitization and intergranular corrosion of tube-to- tubesheet welds of hyper duplex stainless steel using an electrochemical reactivation method. Corrosion Science, 2014. 87: p. 60-70.
7. Swaminathan, J., et al., Sensitization induced stress corrosion failure of AISI 347 stainless steel fractionator furnace tubes. Engineering Failure Analysis, 2011. 18(8): p. 2211-2221.
8. Testing, A. S. f. and Materials, Standard Test Method for Determining the Susceptibility to Intergranular Corrosion of 5XXX Series Aluminum Alloys by Mass Loss After Exposure to Nitric Acid (NAMLT Test). 2004: ASTM International.
9. Zhang, R., et al., A Survey of Sensitization in 5xxx Series Aluminum Alloys. Corrosion, 2016. 72(2): p. 144-159.

1. A method for determining sensitization of an alloy by correlating laser-induced breakdown spectroscopy (LIBS) measurements with the degree of sensitization (DoS) using artificial intelligence (AI), the method comprising:
selective chemical etching of the new phase precipitate of an alloy to induce quantitative chemical composition change on a surface of the alloy, wherein the selective chemical etching is conducted using one or more alloy etchants;
measuring LIBS spectra using a LIBS system to semi-quantitatively probe the chemical composition change on the etched surface of the alloy, wherein the LIBS spectra are measured using a single laser pulse or multiple laser pulses; and
determining sensitization of the alloy using an artificial intelligence (AI) algorithm, wherein the AI algorithm correlates the LIBS spectra with the DoS.

2. The method according to claim 1, wherein the alloy is an aluminum alloy or a steel.

3. The method according to claim 1, wherein a new phase is formed by migration of specific atoms in a crystalline material and is different from the homogeneous alloy.

4. The method according to claim 1, wherein the one or more alloy etchants include one or more of nitric acid, Keller's reagent, and ammonium persulfate.

5. The method according to claim 1, further comprising surface polishing the surface of the alloy before the selective chemical etching.

6. The method according to claim 5, wherein the surface polishing includes one of sanding, ultrasonic polishing, lapping, sandblasting, rumbling and tumbling.

7. The method according to claim 1, wherein the LIBS system includes a single-pulsed laser beam as a plasma excitation source, or two or more pulsed laser beams as the plasme excitation source.

8. The method according to claim 1, wherein the measuring includes detecting a plasma emission during a defined gating interval, with synchronization of each laser pulse.

19. The method according to claim 1, wherein the measuring includes detecting a plasma emission during an integrated time period, without synchronization of each laser pulse.

10. The method according to claim 1, further including implementing a LIBS signal enhancement approach, wherein the LIBS signal enhancement approach includes one of spatial confinement, magnetic confinement, flame enhancement, and argon gas enhancement.

11. The method according to claim 1, wherein the DoS conforms to the standards set by ASTM international standard G67-18 or ASTM international standard G108-94 (2015).

12. The method according to claim 1, wherein the determining sensitization includes providing the quantitative information of the DoS within a range or with a specific value of an alloy.

13. The method according to claim 1, wherein the artificial intelligence algorithm includes one of principal component-discrimination function analysis (PC-DFA), discriminant analysis, partial least squares regression analysis, partial least squares discriminant analysis, a k-nearest neighbors algorithm, an artificial neural network, a soft independent modeling of class analogy, a support vector machine algorithm, and classification and regression trees.

14. The method according to claim 1, wherein the artificial intelligence algorithm includes one of statistical learning, computer intelligence, and soft computing.

15. A method for determining sensitization of an alloy, the method comprising:
selective chemical etching a surface of the alloy, wherein the selective chemical etching is performed using one or more alloy etchants;
measuring laser-induced breakdown spectroscopy (LIBS) spectra of the etched surface of the alloy using a LIBS system to semi-quantitatively probe a chemical composition change of a new phase precipitate of the alloy on the etched surface of the alloy due to the selective chemical etching; and
correlating the LIBS spectra with the degree of sensitization (DoS) of the alloy to thereby determine a sensitization of the alloy.

16. The method of claim 15, wherein the LIBS system includes a single pulse laser source or a multiple pulse laser source.

17. The method of claim 16, wherein the LIBS system further includes one or more compact or bulk spectrometers, and optical components for light delivery and collection.

18. The method according to claim 15, wherein the alloy is an aluminum alloy a steel.

19. The method according to claim 15, further comprising surface polishing the surface of the alloy before the selective chemical etching.

20. The method according to claim 15, wherein the correlating includes using an artificial intelligence (AI) algorithm to determine the sensitization of the alloy.

21. The method of claim 20, wherein the artificial intelligence algorithm includes one of principal component-discrimination function analysis (PC-DFA), discriminant analysis, partial least squares regression analysis, partial least squares discriminant analysis, a k-nearest neighbors algorithm, an artificial neural network, a soft independent modeling of class analogy, a support vector machine algorithm, and classification and regression trees.

22. A laser-induced breakdown spectroscopy (LIBS) system for determining sensitization of an alloy, the system comprising:
a pulsed laser source configured to emit laser pulses directed at a sample to induce a plasma on a surface of the sample, the sample including a selectively chemical etched surface of an alloy;
a spectrometer configured to measure LIBS spectra of the etched surface of the alloy to semi-quantitatively probe a chemical composition change of a new phase precipitate of the alloy on the etched surface of the alloy; and
one or more processors, configured to correlate the LIBS spectra with a degree of sensitization (DoS) of the alloy to thereby determine a sensitization of the alloy.

23. The system of claim 22, wherein the one or more processors are configured to correlate using an artificial intelligence (AI) algorithm to determine the sensitization of the alloy.

24. The system of claim 23, wherein the artificial intelligence algorithm includes one of principal component-discrimination function analysis (PC-DFA), discriminant analysis, partial least squares regression analysis, partial least squares discriminant analysis, a k-nearest neighbors algorithm, an artificial neural network, a soft independent modeling of class analogy, a support vector machine algorithm, and classification and regression trees
